(19) United States
(12) Patent Application Publication
(10) Pub. No.: US 2026/0216034 A1
(43) Pub. Date: Jul. 30, 2026

(54) NON-THERAPEUTIC USE OF A COMPOSITION COMPRISING OLEUROPEIN

(71) Applicant: BIOACTOR BV, Maastricht (NL)

(72) Inventors: Antonie Johannes VAN DER SAAG, Maastricht (NL); Yala STEVENS, Maastricht (NL); Maria IMPERATRICE, Maastricht (NL)

(73) Assignee: BIOACTOR BV, Maastricht (NL)

(21) Appl. No.: 19/120,573

(22) PCT Filed: Oct. 12, 2023
(86) PCT No.: PCT/EP2023/078317

§ 371 Date: Apr. 11, 2025

(30) Foreign Application Priority Data
Oct. 12, 2022 (EP) 22201085.2

(51) Int. Cl. A61K 8/60; A61Q 19/08

(52) U.S. Cl. CPC A61K 8/602; A61Q 19/08; A61K 2800/92

(57) ABSTRACT

The present invention relates to the field of food supplementation, in particular to the field of nutraceuticals, more in particular to the use of a composition comprising oleuropein. More specifically, the present invention relates to the non-therapeutic use of a composition comprising oleuropein or a metabolite thereof for reducing (post-)menopausal symptoms, improving body composition parameters, improving skin-aging parameters, and/or improving muscle strength parameters in a human subject.

### CROSS-REFERENCE TO RELATED APPLICATIONS

This application is a national stage application under 35 U.S.C. § 371 of International Application No. PCT/EP2023/078317, filed Oct. 12, 2023, which International Application claims benefit of priority to European Patent Application No. 22201085.2, filed Oct. 12, 2022, each of which is incorporated herein by reference in its entirety.

### FIELD OF THE INVENTION

The present invention relates to the field of food supplementation, in particular to the field of nutraceuticals, more in particular to the use of a composition comprising oleuropein. More specifically, the present invention relates to the non-therapeutic use of a composition comprising oleuropein or a metabolite thereof for reducing (post-)menopausal symptoms, improving body composition parameters, improving skin-aging parameters, and/or improving muscle strength parameters in a human subject.

### BACKGROUND TO THE INVENTION

The most popular fat in the Mediterranean diet is olive oil. Its beneficial effects are attributed to its unique fatty acid profile and the presence of a number of specific components such as oleuropein, the main phenolic component present in the olive tree. Oleuropein is an ester of hydroxytyrosol, which is present in large quantities in the leaves of the olive tree and can be extracted therefrom. Oleuropein is hydrolysed in the digestive system to hydroxytyrosol which, in turn, is absorbed into the blood. Oleuropein was identified as the most suitable precursor of hydroxytyrosol for incorporation into foods due to its greater stability during digestion and consequently its higher bioavailability.

Experimental and clinical studies show beneficial health effects of olive leaf extracts containing oleuropein. Compositions comprising an amount of oleuropein have primarily an antioxidant and anti-inflammatory effects and in particular a bone-protective effect. In this respect, it was found that oleuropein is protective against bone loss induced or exacerbated by estrogen deficiency, oxidative stress and/or chronic inflammation (WO2009132807A1). Furthermore, it is known that a composition comprising oleuropein can be used for improving a physiological state linked to metabolic fatigue in cells by boosting mitochondrial function (WO2020229538A1/WO2022106408) or to improve or maintain muscle quality or muscle mass in an individual (WO2019101700). Therefore, oleuropein may be a promising non-therapeutic candidate for use in other aging-related related conditions.

Normal aging in human subjects—but certainly the (post-)menopausal (transition) period-coincides with changes in many tissues going from muscle to skin which are mostly due to changes in the hormonal balance. Aging and the (post-)menopausal status are associated with (i) skin-aging which clinically manifests as increased roughness, wrinkle formation, mottled pigmentation or even precancerous skin changes; (ii) changes in the body composition such as abdominal adipose tissue accumulation, increase in weight and waist circumference, etc. and (iii) loss of muscle mass or muscle atrophy which causes a decreased muscle strength (sarcopenia). Moreover, during (post)-menopause, women may experience other physical symptoms such as sleep disturbance, urogenital problems and emotional symptoms such as mood swings, which have a significant impact on their health.

Modern science has developed several drugs for treating (chronic) diseases or alleviating certain associated symptoms by targeting specific molecular mechanisms. These drugs are usually enormously expensive and are associated with serious side effects and morbidity. For example, medical treatments for (post)-menopausal symptoms are available, including hormone replacement therapy (HRT). However, such treatment is associated with side effects as exemplified by the Million Women Study which highlighted the correlation between estrogen-progestogen replacement therapy and increased incidence of breast cancer (Beral et al. 2003). Moreover, HRT intake has been correlated to a higher rate of depressive symptoms in Dutch menopausal women (Maartens et al. 2000). Therefore, the interest in more natural-alternative therapies to counteract the side effect symptoms has grown. No such medication or compositions exists that are directed to alleviate or improve symptoms or problems coinciding with aging or (post-) menopause. Plant products and plant-derived nutraceuticals such as oleuropein provide such a solution to this problem. The beneficial effect of an olive oil extract composition on breast and ovarian cancer, post-menopausal osteoporosis, cardiovascular disease and Type 2 Diabetes have been already evaluated (Ly et al. 2021). The use of oleuropein supplementation to reduce or improve a broader spectrum of aging-related and (post-)menopausal symptoms, such as change in body composition, skin-aging and menopause-related quality of life is not known.

Therefore, it is an object of the present invention to provide a non-therapeutic use of a composition comprising oleuropein or a metabolite thereof for reducing (post-)menopausal symptoms, improving body composition parameters, improving skin-aging parameters, and/or improving muscle strength parameters in a human subject.

### SUMMARY OF THE INVENTION

In a first aspect, the present invention provides the non-therapeutic use of a composition comprising oleuropein or a metabolite thereof for reducing (post-)menopausal symptoms, improving body composition parameters, improving skin-aging parameters, and/or improving muscle strength parameters in a human subject.

In a further embodiment, the (post-)menopausal symptoms are one or more symptoms selected from the list comprising: night sweats, mood swings, tearfulness, bloating, fatigue, physical inactivity, hormonal imbalance, sleep disturbance, weight gain, thinning hair, nail brittle, hot flashes, chills, irregular periods, headaches, anxiety, breast soreness, decreased libido, vaginal dryness or burning, genital or vulvovaginal atrophy, dysuria, satiating capacity, dietary habits, and/or muscle atrophy.

In another embodiment, the body composition parameters are one or more parameters selected from the list comprising: waist circumference, calf circumference, arm circumference, hip circumference, leg circumference, trunk circumference, abdominal circumference, appendicular lean mass, weight, height, total body fat free mass, fat mass, bone mineral density and/or visceral abdominal tissue.

In yet another embodiment, the skin-aging parameters are one or more parameters selected from the list comprising: skin elasticity skin moisturization, skin firmness, trans epidermal water loss, skin hydration, skin radiance, overall skin quality, skin sebum content, skin cellular oxidative stress and/or wrinkle depth.

In yet another embodiment, the muscle strength parameters are one or more parameters selected from the list comprising: handgrip strength, muscle thermogenesis, and/or muscle contraction.

In a specific embodiment, the composition according to the invention comprises a standardized amount of oleuropein of about and between 5% to 70% (w/v), preferably about and between 10% to 50% (w/V), more preferably about and between 20% to 40% (w/v), most preferably 40% (w/V).

In another specific embodiment, the composition comprises at least 20 mg, preferably at least 30 mg, more preferably at least 100 mg of oleuropein, most preferably at least 125 mg of oleuropein.

In yet another specific embodiment, the composition is formulated in a daily dosage form comprising 1 to 15 mg/kg, in particular 1 to 3 mg/kg of oleuropein.

In a particular embodiment, the composition comprises oleuropein in combination with one or more additional compounds selected from a group consisting of: verbascoside, caffeine, taurine, theanine, inositol, guarana, magnesium, melatonin, ribose, carnitine, co-enzym Q10, alpha-glycerophosphocholine, alpha-lipoic acid, luteolin-7-glucoside, luteolin, quercetin, and xylose, in particular verbascoside.

In another particular embodiment, the amount of verbascoside in said composition is about and between 0.1% to 5.0% (w/v), more in particular about and between 0.5% to 2.5% (w/v), in particular 1.0% (w/V).

In an embodiment, the metabolite of oleuropein is selected from the group comprising oleuropein aglycone, hydroxytyrosol, homovanillyl alcohol, isohomovanillyl alcohol, glucuronidated forms thereof, sulfated forms thereof, derivatives thereof, and mixtures thereof.

In a specific embodiment, the composition further comprises a solvent selected from the list comprising:
water or ethanol or a combination thereof.

In a further embodiment, the human subject has an age of more than 45 years.

In yet a further embodiment, the human subject is a (post-)menopausal woman.

In a particular embodiment, the oleuropein is extracted from the leaves of a plant belonging to the Oleaceae family.

In a particular embodiment, the composition is an oral composition.

In yet a particular embodiment, the composition is in the form of a pill, powder, capsule, tablet, gel, beverage, chewing gum, chewable tablet, lozenge or troche.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Body composition after 4 weeks of supplementation in overweight/obese subjects. (A) Reduction in waist circumference after 4 weeks of olive leaf extract supplementation compared to baseline (p=0.099); (B) lower waist circumference in group which received olive leaf extract compared to the placebo group (p=0.22).

FIG. 2: Self-assessed post-menopause symptoms scores at baseline, after 14 days supplementation and after 28 days of supplementation. (A) night sweats, (B) feeling bloated, (C) feelings of happiness, (D) feeling relaxed, (E) feeling tearful easily, (F) mood swings, (G) energy, (H) vitality, (I) overall hair quality, (J) nail strength, (K) overall skin quality, (L) skin moisturization (M) skin elasticity. Data are expressed as means on a 10-point Likert scale ranging from a minimum of 1 “very low/bad” till a maximum of 10 “very high/good”. * Sig (P≤0.05) baseline vs 14 days; ** Sig (P≤0.05) baseline vs after 28 days.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

The overall objective of the present invention is to provide a composition for non-therapeutic use to reduce, alleviate, improve symptoms related with normal aging but in particular, symptoms that are apparent before or during the (post-)menopausal period, including symptoms of the skin, body composition or appearance, muscle capacity as well as mental state of subjects. These symptoms are induced or exacerbated by hormonal disturbance, estrogen deficiency, oxidative stress and/or chronic inflammation. Where the composition of the present invention are particularly suitable for use in (post-) menopausal women, they may also be suitably used in non-menopausal women or men, in particular for use in improving body composition parameters, improving skin-aging parameters, and/or improving muscle strength.

As already mentioned hereinbefore, it was surprisingly found that a composition comprising oleuropein has beneficial effects on body composition parameters, skin-aging parameters, and muscle strength parameters. In particular, as described in example 2 of the present invention, a reduction of +1 cm in waist circumference was observed in 39 subjects with overweight or obesity who were supplemented with an oleuropein extract for 4 weeks. Furthermore, symptoms related with (post-) menopause were alleviated in women aged 45 and beyond (as detailed in example 3), more in particular after 28 days of supplementation a reduction was perceived in night sweats, mood swings and tearfulness while overall perception of energy, vitality, hair and skin quality, nail strength improved.

In a first aspect, the present invention provides the non-therapeutic use of a composition comprising oleuropein or a metabolite thereof for reducing (post-)menopausal symptoms, improving body composition parameters, improving skin-aging parameters, and/or improving muscle strength parameters in a human subject.

In the context of the present invention, the term ‘non-therapeutic use’ is to be understood as a non-therapeutic method to alleviate, improve or reduce symptoms that are related with a particular disease, illness or ailment, without the actual treatment or prevention of the disease as such. Substances or compounds can be used non-therapeutically in the context of cosmetic treatment. In the context of the present invention, the non-therapeutic use can also mean the use of substances or composition for purposes which cannot be properly understood as therapeutic, for example in the following circumstances:
a) where there is a disease or some form of ill health which has been correctly diagnosed and for which no pharmacological treatment is available;
b) where there is no specific and authorized prescription for drugs;
c) where the substance is used at dosages, in ways and for time periods which are in line with what has been established by scientific research as ‘effective and tolerable’ in to alleviate, reduce, or improve symptoms that are associated with an individual's particular illness.

As an example, a composition comprising oleuropein can be administered to an obese person in an effective dosage, and optionally repeating said dosage, until a beneficial loss of body weight has occurred, without specifically treating obesity. Accordingly, the beneficial effect can also be a cosmetically beneficial effect such as for example reduced waist circumference.

As used herein, an “effective dosage” is an amount that prevents a deficiency, ameliorates a disease or medical condition in an individual or, more generally, reduces symptoms, manages progression of the diseases or provides a nutritional, physiological, or medical benefit to the individual. The relative terms “improved,” “increased,” “enhanced”, “alleviated”, “reduce”, “ameliorate” and the like refer to the effects of the composition disclosed herein, namely a composition comprising oleuropein or a metabolite thereof, relative to a composition lacking oleuropein or a metabolite thereof but otherwise identical (i.e. placebo or control). For example, a composition according to the invention can reduce or improve disclosed symptoms by at least 5%, such as at least 10%, at least 15%, at least 20%, preferably at least 25%, more preferably at least 30%, and particularly preferably at least 40%, when compared to untreated subjects. In another example, composition according to the invention can reduce or improve disclosed symptoms by at least 0.1%, such as at least 0.5%, at least 1.0%, at least 1.5%, preferably at least 2.0%, more preferably at least 2.5%, and particularly preferably at least 3.0 when compared to untreated subjects.

A particular object of the present invention relates to the non-therapeutic use of a composition comprising oleuropein or a metabolite thereof for reducing, alleviating, improving (post-)menopausal symptoms.

In the context of the present invention, the term ‘menopause’ is to be understood as the permanent cessation of menstruation resulting in the loss of ovarian follicle development. Menopause usually occurs between the age of 47 and 54 when a woman has not had any menstruation for 12 months. Menopause may also be defined by a decrease in hormone production by the ovaries. Women may experience hot flashes, shivering, sweating, and reddening of the skin. Other symptoms may include vaginal dryness, trouble sleeping, reduced nail strength or nail brittle, and mood swings. The severity of symptoms varies between women. As used herein, the term menopause also includes the ‘menopausal transition’ or ‘perimenopause’ and is to be understood as the period of time beginning with the onset of irregular menstrual cycles until the last menstrual period, and is marked by fluctuations in reproductive hormones. The menopausal transition is characterized by variable cycle lengths and missed menses. In the context of the present invention, the term ‘post-menopause’ is to be understood as the years of a woman's life after menopause occurs. While menopause is defined retrospectively as the time of the final menstrual period, followed by 12 months of amenorrhea, post-menopause describes the period following the final menses.

In particular, the (post-)menopausal symptoms as defined herein, relate to symptoms that can occur during the perimenopause, menopause and/or post-menopause. The subdivision is not intended to make a difference between symptoms but as an indication that menopause is associated with a variety of symptoms over a relative long timespan. Thus, when referring to menopause symptoms, it may also refer to symptoms occurring during the perimenopause or post-menopause period.

In a further embodiment, the (post-)menopausal symptoms are one or more symptoms selected from but not limited to the list comprising: night sweats, mood swings, tearfulness, bloating, fatigue, physical inactivity, hormonal imbalance, sleep disturbance, weight gain, thinning hair, nail brittle, hot flashes, chills, irregular periods, headaches, anxiety, breast soreness, decreased libido, vaginal dryness or burning, genital or vulvovaginal atrophy, dysuria, satiating capacity, dietary habits, and/or muscle atrophy.

In another particular embodiment, the present invention relates to the non-therapeutic use of a composition comprising oleuropein or a metabolite thereof for reducing, alleviating, improving (post-) menopausal symptoms selected from the list comprising: painful intercourse, lack of energy, joint soreness, stiffness, back pain, breast enlargement, heart palpitations, dizziness, dry, itchy skin, thinning, tingling skin, rosacea, urinary incontinence, urinary urgency, interrupted sleeping patterns, poor memory, inability to concentrate, depressive mood, irritability, mood swings, and less interest in sexual activity.

(Post-)menopausal symptoms can be assessed using questionnaires and are disclosed in the method section.

In a particular embodiment, the use of a composition comprising oleuropein or a metabolite thereof can improve or reduce at least one (post)-menopausal symptom selected from the above-mentioned list by at least 0.1%, such as at least 0.5%, at least 1.0%, at least 1.5%, preferably at least 2.0%, more preferably at least 2.5%, and particularly preferably at least 3.0 when compared to untreated subjects.

In another particular embodiment, the use of a composition comprising oleuropein or a metabolite thereof can improve or reduce at least one (post)-menopausal symptom selected from the above-mentioned list of symptoms by at least 5%, such as at least 10%, at least 15%, at least 20%, preferably at least 25%, more preferably at least 30%, and particularly preferably at least 40%, when compared to untreated subjects.

For example, the use of a composition comprising oleuropein or a metabolite thereof can improve hormonal balance in (post-)menopausal women by 5%, 10%, 15%, 20%, 25%, 30%, 40% compared to subjects who did not receive a composition according to the invention.

The above-described non-therapeutic use of the composition according to the invention is specifically directed to women experiencing (post-)menopausal symptoms. However, the composition described herein below can also be applied in other segments of the population being men, or women that are not in the (post-)menopausal period.

As such, another object of the present invention relates to the non-therapeutic use of a composition comprising oleuropein or a metabolite thereof for reducing, alleviating, improving body composition parameters.

In the context of the present invention, ‘body composition” is to be understood as a term that describes the percentages of fat, bone, water, and muscle in the human body. Besides traditional methods such as weight or BMI, it provides a more complete indication of your overall health.

In another embodiment, the body composition parameters are one or more parameters selected from but not limited to the list comprising: waist circumference, calf circumference, arm circumference, hip circumference, leg circumference, trunk circumference, abdominal circumference, appendicular lean mass, weight, height, total body fat free mass, fat mass, bone mineral density and/or visceral abdominal tissue.

Body composition parameters can be measured by different techniques such as dual energy X-ray absorptiometry (DEXA) but is not limited thereto. DEXA is a “Gold Standard” in body composition testing and is highly reproducible. Total body scans using DEXA give accurate and precise measurements of body composition, including bone mineral content, bone mineral density, lean tissue mass, fat tissue mass, and fractional contribution of fat. Other suitable techniques to measure body composition parameters can be air displacement plethysmography (ADP) bioelectrical impedance analysis (BIA) or body volume indicator (BVI).

In a particular embodiment, the use of a composition comprising oleuropein or a metabolite thereof can improve or reduce at least one body composition parameter selected from the above-mentioned list by at least 0.1%, such as at least 0.5%, at least 1.0%, at least 1.5%, preferably at least 2.0%, more preferably at least 2.5%, and particularly preferably at least 3.0% when compared to untreated subjects.

In another particular embodiment, the use of a composition comprising oleuropein or a metabolite thereof can improve or reduce at least one body composition parameter selected from the above-mentioned list by at least 5%, such as at least 10%, at least 15%, at least 20%, preferably at least 25%, more preferably at least 30%, and particularly preferably at least 40%, when compared to untreated subjects.

Still another object of the present invention relates to the non-therapeutic use of a composition comprising oleuropein or a metabolite thereof for reducing, alleviating, improving skin-aging parameters.

In the context of the present invention, the term ‘skin” refers to any part of the body and/or face, including the scalp. Common changes in the skin as a result of aging range from wrinkles, discoloration, and skin laxity but can manifest in more severe forms such as skin malignancies. As used herein, the term ‘photo aging’ refers to a process wherein changes in the skin are accelerated and/or exacerbated by sun exposure.

In a particular embodiment, the skin-aging parameters are one or more parameters selected from but not limited to the list comprising: skin elasticity skin moisturization, skin firmness, trans epidermal water loss, skin sebum content, skin cellular oxidative stress and/or wrinkle depth.

Skin-aging parameters can be measured noninvasively and are disclosed in the method section.

In a particular embodiment, the use of a composition comprising oleuropein or a metabolite thereof can improve or reduce at least one skin-aging parameter selected from the above-mentioned list by at least 0.1%, such as at least 0.5%, at least 1.0%, at least 1.5%, preferably at least 2.0%, more preferably at least 2.5%, and particularly preferably at least 3.0 when compared to untreated subjects.

In another particular embodiment, the use of a composition comprising oleuropein or a metabolite thereof can improve or reduce at least one skin-aging parameter selected from the above-mentioned list by at least 5%, such as at least 10%, at least 15%, at least 20%, preferably at least 25%, more preferably at least 30%, and particularly preferably at least 40%, when compared to untreated subjects.

Another object of the present invention relates to the non-therapeutic use of a composition comprising oleuropein or a metabolite thereof for reducing, alleviating, improving muscle strength parameters.

In the context of the present invention, the term ‘muscle strength’ is to be understood as the amount of force a muscle is able to produce and is measured by the maximum amount of force a muscle can produce in a single effort (maximal effort). For example, during the isometric hand-grip strength, subjects need to exert maximal force during consecutive measurements which results in a valid surrogate measure of overall muscular strength. As used herein, muscle strength also correlates with muscle loss and thus also the decline in muscle strength associated with aging and/or menopause.

In another embodiment, the muscle strength parameters are one or more parameters selected from but not limited to the list comprising: handgrip strength, muscle thermogenesis, and/or muscle contraction.

In a particular embodiment, the use of a composition comprising oleuropein or a metabolite thereof can improve or reduce at least one muscle strength parameter selected from the above-mentioned list by at least 0.1%, such as at least 0.5%, at least 1.0%, at least 1.5%, preferably at least 2.0%, more preferably at least 2.5%, and particularly preferably at least 3.0 when compared to untreated subjects.

In another particular embodiment, the use of a composition comprising oleuropein or a metabolite thereof can improve or reduce at least one muscle strength parameter selected from the above-mentioned list by at least 5%, such as at least 10%, at least 15%, at least 20%, preferably at least 25%, more preferably at least 30%, and particularly preferably at least 40%, when compared to untreated subjects.

Within the context of the present invention, the term “subject” or “individual” or “participant” refers to a human, in particular an older adult, more in particular an elderly. Preferentially, the subject is a human subject wherein the above defined aging and/or (post-)menopausal symptoms are perceptible or at least are experienced by the subject. The term “older adult” in the context of a human means an age from birth of at least 45 years, preferably above 50 years, more preferably above 55 years, and includes elderly individuals. The term “elderly” in the context of a human means an age from birth of at least 60 years, preferably above 63 years, more preferably above 65 years, and most preferably above 70 years.

In a further embodiment, the human subject has an age of more than 45 years.

In another embodiment, human subject has an age of at least 45 years such as at least 50 years, at least 55 years, at least 60 years, at least 65 years, at least 70 years, at least 75 years.

As described herein, the composition according to the invention can be used to alleviate, reduce or improve symptoms relating to body composition, skin aging, muscle strength and thus be applied to a broad segment of the population including men and women experiencing or perceiving such aging symptoms.

In a particular embodiment, the human subject is a woman.

In yet a further embodiment, the human subject is a (post-)menopausal woman.

In yet another embodiment, the human subject is a (post-)menopausal woman with an age of at least 45 years.

The composition according to the invention is particularly useful for elderly women in the (post-) menopause. More in particular, the composition according to the invention is particularly suitable to reduce, alleviate or improve (post-)menopausal symptoms, skin-aging symptoms, body composition symptoms and/or muscle strength symptoms as defined herein.

In another embodiment, the compositions according to the invention may further comprise a combination of extracts with a standardized amount of oleuropein. In particular, such extract is typically an extract from leaves, stems, fruits and/or stones; more in particular from the leaves of plants belonging to the Oleaceae family. Plants belonging to the Oleaceae family include a plant of genus Olea europaea (olive tree), a plant of genus Ligustrum, a plant of genus Syringa, a plant of genus Fraximus, a plant of genus Jasminum and a plant of genus Osmanthus.

In a particular embodiment, the oleuropein is extracted from the leaves of a plant belonging to the Oleaceae family. In another particular embodiment, oleuropein is extracted from the leaves of a plant of the genus Olea europaea (olive tree).

In a specific embodiment, the composition according to the invention comprises a standardized amount of oleuropein of about and between 5% to 70% (w/v), preferably about and between 10% to 50% (w/V), more preferably about and between 20% to 40% (w/v), most preferably 40% (w/V).

According to a particular embodiment, the composition may comprise a standardized amount of oleuropein equal to or at least 5% (w/v); such as equal to or at least 10%, 15%, 20%, 25%, 30%, 35%, 40% (w/V).

In an embodiment, the present invention provides the non-therapeutic use of a composition comprising a standardized amount of at least 10% (w/v), such as at least 20% (w/v), at least 30% (w/v), in particular 40% (w/v) oleuropein for reducing (post-)menopausal symptoms in a human subject.

In another embodiment, the present invention provides the non-therapeutic use of a composition comprising a standardized amount of at least 10% (w/v), such as at least 20% (w/v), at least 30% (w/v), in particular 40% (w/v) oleuropein for improving body composition parameters in a human subject.

In another embodiment, the present invention provides the non-therapeutic use of a composition comprising a standardized amount of at least 10% (w/v), such as at least 20% (w/v), at least 30% (w/V), in particular 40% (w/v) oleuropein for improving skin-aging parameters in a human subject.

In another embodiment, the present invention provides the non-therapeutic use of a composition comprising a standardized amount at least 10% (w/v), such as at least 20% (w/v), at least 30% (w/V), in particular 40% (w/v) oleuropein for improving muscle strength parameters in a human subject.

Exemplary methods for preparing oleuropein rich leaf extracts derived from olives may be found in U.S. Pat. No. 6,676,980. As an illustrative example, oleuropein rich leaf extracts are prepared using a method comprising the following steps:
a) immersing Olea europaea leaves with an extraction solution being water or an extraction solution essentially made of an alcoholic solution comprising at least 25% alcohol by weight, for example ethanol, for a period of time of at least 4 hours;
b) recovering the resultant extraction solution from end of step a);
c) immersing de novo Olea europaea leaves from step a) with a new extraction solution, which composition is similar to that defined hereabove, for a period of time of at least 4 hours;
d) recovering the extraction solution from end of step c);
e) collecting the resultant extraction solutions respectively from step b) and step c);
f) vacuum distillating the resultant solution from step e), for example at 70° C., so as to yield a concentrate comprising from 30% to 40% by weight of solids; and
g) drying the resultant concentrate from end of step f, for example by spraying at 70° C., so as to yield an extract as particles comprising from 30% to 40% by weight of the oleuropein compound.

In a specific embodiment, the composition comprises at least about 25%, at least about 35%, at least about 45%, at least about 55%, at least about 65%, at least about 75% alcohol by weight.

In a specific embodiment, the composition further comprises a solvent selected from the list comprising: water or ethanol or a combination thereof.

Other alcohols that are miscible with water that are known in the art are also suitable as solvent and may include, but not limited to methanol, n-propyl alcohol, isopropyl alcohol, and t-butyl alcohol.

In an embodiment of the present invention, the compositions are prepared from extracts of immature or semi-mature olives. Olives are known to be rich in polyphenolics, notably oleuropein and hydroxytyrosol.

In an embodiment, the metabolite of oleuropein is selected from the group comprising oleuropein aglycone, hydroxytyrosol, homovanillyl alcohol, isohomovanillyl alcohol, glucuronidated forms thereof, sulfated forms thereof, derivatives thereof, and mixtures thereof.

Although the composition according to the invention essentially comprises oleuropein, some percentages of oleuropein metabolite(s) may be present. Visa versa, in a composition essentially comprising one or more metabolites of oleuropein, some percentages of oleuropein may be present.

Therefore, the invention also provides a composition comprising oleuropein and a metabolite thereof selected from the group comprising oleuropein aglycone, hydroxytyrosol, homovanillyl alcohol, isohomovanillyl alcohol, glucuronidated forms thereof, sulfated forms thereof, derivatives thereof, and mixtures thereof.

In a specific embodiment, the composition according to the invention comprises oleuropein in combination with aglycone; alternatively oleuropein in combination with hydroxytyrosol; alternatively oleuropein in combination with homovanillyl alcohol; alternatively oleuropein in combination with isohomovanillyl alcohol; alternatively oleuropein in combination with aglycone, hydroxytyrosol; alternatively oleuropein in combination with aglycone, homovanillyl alcohol; alternatively oleuropein in combination with aglycone, isohomovanillyl alcohol; alternatively oleuropein in combination with hydroxytyrosol, homovanillyl alcohol; alternatively oleuropein in combination with hydroxytyrosol, isohomovanillyl alcohol; alternatively oleuropein in combination with homovanillyl alcohol, isohomovanillyl alcohol; alternatively oleuropein in combination with aglycone, hydroxytyrosol, homovanillyl alcohol; alternatively oleuropein in combination with aglycone, hydroxytyrosol, isohomovanillyl alcohol; alternatively oleuropein in combination with aglycone, homovanillyl alcohol, isohomovanillyl alcohol; alternatively oleuropein in combination with hydroxytyrosol, homovanillyl alcohol, isohomovanillyl alcohol; alternatively oleuropein in combination with aglycone, hydroxytyrosol, homovanillyl alcohol, isohomovanillyl alcohol.

In a particular embodiment, the composition according to the invention comprises oleuropein and a metabolite selected from one of the combinations defined above in a weight ratio of oleuropein:metabolite equal to or greater than 1:1, in particular about 2:1 and up to about 100:1; in particular between about 1:1 and about 50:1; more in particular between about 1:1 and about 10:1; even more in particular about 1:1 and about 4:1; in particular a ratio of about 2:1.

In a particular embodiment, the composition according to the invention comprises oleuropein and a metabolite selected from one of the combinations defined above in a weight ratio of oleuropein: metabolite equal to or greater than 1:1, in particular at least about 2:1, such as at least 3:1, at least 4:1, at least 10:1, at least 15:1, at least 20:1, at least 25:1 at least 50:1, at least 75:1, at least 100:1, at least 200:1.

In a specific embodiment, the composition according to the invention comprises oleuropein and hydroxytyrosol, in a weight ratio of oleuropein:hydroxytyrosol equal to or greater than 1:1, in particular about 2:1 and up to about 100:1; in particular between about 1:1 and about 50:1; more in particular between about 1:1 and about 10:1; even more in particular about 1:1 and about 4:1; in particular a ratio of about 2:1.

In a specific embodiment, the composition according to the invention comprises oleuropein and hydroxytyrosol, in a weight ratio of oleuropein:hydroxytyrosol equal to or greater than 1:1, in particular at least about 2:1, such as at least 3:1, at least 4:1, at least 10:1, at least 15:1, at least 20:1, at least 25:1 at least 50:1, at least 75:1, at least 100:1, at least 200:1.

Said compositions comprising an excess of oleuropein of at least about 2:1, such as at least 4:1; are particularly useful for non-therapeutic use for reducing (post-)menopausal symptoms, improving body composition parameters, improving skin-aging parameters, and/or improving muscle strength parameters in a human subject.

In some embodiments, the compositions according to the invention comprises between and about 5 mg to 1 g of oleuropein and/or one or more metabolite thereof; in particular comprising between and about 10 mg to 500 mg; more in particular comprising between 20 mg to 200 mg; even more in particular comprising between 30 mg to 150 mg of oleuropein and/or one or more metabolite thereof.

In another embodiment, the composition comprises at least 20 mg, preferably at least 30 mg, more preferably at least 100 mg of oleuropein, most preferably at least 125 mg of oleuropein and/or one or more metabolite thereof.

In an even further embodiment, the composition according to the invention comprises oleuropein and/or one or more metabolite thereof; between and about 5 mg to 1 g; in particular between and about 10 mg to 500 mg; more in particular between and about 20 mg to 200 mg, even more in particular between and about 30 mg to 150 mg.

In another embodiment, the composition according to the invention comprises oleuropein and hydroxytyrosol; between and about 5 mg to 1 g of each of the polyphenols hydroxytyrosol and oleuropein; in particular comprising between and about 10 mg to 500 mg of each of the polyphenols hydroxytyrosol and oleuropein; more in particular comprising between 20 mg to 200 mg of each of the polyphenols hydroxytyrosol and oleuropein; even more in particular comprising about 30 mg of hydroxytyrosol and about 150 mg of oleuropein.

The effective amount of a composition, according to the present invention, will of course vary with the particular composition, the route of administration, the age and condition of the recipient, and the symptoms of the particular disorder or disease. For a typical administration, a daily dosage of about 0.5 to 15 mg/kg; in particular 0.5 to 3 mg/kg of oleuropein can be applied. For a typical person of 50-80 kg, this would require an intake of about 25-500 mg per day of oleuropein; in particular 25-250 mg per day. A non-limiting example of an embodiment comprises daily administration of a composition comprising about 125 mg oleuropein or metabolite thereof. Another example can be daily administration of a composition comprising about 250 mg of oleuropein or metabolite thereof.

In an embodiment, the composition according to the invention can be administered in multiple doses per day and more specifically at least once, twice, three times, four times a day, preferably in one or two doses per day. In another embodiment, the composition can also be administered on a regimen of between one and four intakes per day.

In a preferred embodiment, the composition is administered on a regimen of two intake per day.

In a particular embodiment, the composition is administered on a regimen of two intake per day wherein each intake comprises about and between 20 mg to 50 mg oleuropein or metabolite thereof.

In another particular embodiment, the composition is administered on a regimen of two intake per day wherein each intake comprises about and between 100 mg to 150 mg, preferably 125 mg of oleuropein or metabolite thereof.

In a specific embodiment, the composition is formulated in a daily dosage form comprising 1 to 15 mg/kg, in particular 1 to 3 mg/kg of oleuropein. In a very specific embodiment, the composition is formulated in a daily dosage form comprising about 1, 2, 3, 4, 5, 6, 7, 8, 9, 10, 11, 12, 13, 14, 15 mg/kg of oleuropein.

In another embodiment, the composition is formulated in a daily dosage form comprising oleuropein and hydroxytyrosol between and about 1 to 15 mg/kg, in particular between and about 1 to 3 mg/kg of each of hydroxytyrosol and oleuropein. In a very specific embodiment, the composition is formulated in a daily dosage form comprising oleuropein and hydroxytyrosol of about 1, 2, 3, 4, 5, 6, 7, 8, 9, 10, 11, 12, 13, 14, 15 mg/kg of each of hydroxytyrosol and oleuropein.

The composition is administered as a supplement to the diet of an individual multiple times a week, more specifically once, twice, three times, four times, five times, six times, seven times a week, preferably daily.

In an embodiment, the composition is administered to the individual consecutively for a number of days until a beneficial effect is achieved. For example, the composition can be administered to the individual daily for at least 10, 20, 30, 40, 50, 60, 70, 80, 90, 100, 110, 120 or more consecutive days. As another example, the composition can be administered to the individual for a longer period, such as a period of 1, 2, 3, 4, 5, 6, 7, 8, 9 or 10 years.

In a particular embodiment, the composition is administered to the individual for at least 2 week, such as at least 3 weeks, at least 4 weeks, preferably at least 2 weeks.

In another embodiment, the composition is administered at least 3 months, for example a period of 3 months to 1 year, and preferably for at least 6 months.

The above examples of administration do not require continuous daily administration with no interruptions. Instead, there may be some short breaks in the administration, such as a break of two to four days during the period of administration. The ideal duration of the administration of the composition can be determined by those of skill in the art.

In some embodiments, the compositions may also comprise optionally one or more of a protein source, a free amino acid, a carbohydrate source, a fat source, a vitamin, or a mineral.

In a particular embodiment, the composition comprises oleuropein in combination with one or more additional compounds selected from a group consisting of: verbascoside, caffeine, taurine, theanine, inositol, guarana, magnesium, melatonin, ribose, carnitine, co-enzym Q10, alpha-glycerophosphocholine, alpha-lipoic acid, luteolin-7-glucoside, luteolin, quercetin, and xylose, in particular verbascoside.

Leaves and drupes from the olive tree, Olea europaea, are rich in biophenols such as oleuropein, verbascoside, ligstroside, tyrosol, and hydroxytyrosol, which have shown wide antioxidant and antimicrobial properties. As used herein, verbascoside is a natural molecule belonging to the extensive family of phenylpropanoids. Verbascoside is the main hydroxycinnamic derivative in olives, and it increases during the fruit maturation. It seems that there is a reverse relationship between oleuropein and verbascoside; samples that have high levels of verbascoside also have low levels of oleuropein, and vice versa. Several biological properties have been described, such as anti-inflammatory, antimicrobial, antitumor and antioxidant and has beneficial effects in reducing pain, oral mucositis score, burning and erythema.

In another particular embodiment, the amount of verbascoside in said composition is about and between 0.1% to 5.0% (w/v), more in particular about and between 0.5% to 2.5% (w/v), in particular 1.0% (w/V).

In another particular embodiment, the amount of verbascoside in said composition may be at least about 0.1%, such as at least 0.5%, at least 1.0%, at least 1.5%, at least 2.0%, at least 2.5%, at least 2.5% (w/V).

In a particular embodiment, the amount of verbascoside is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5% (w/v), preferably 1.0% (w/v).

The compositions for non-therapeutic use of the present invention, can be prepared in any known or otherwise effective dosage or product form suitable for use in providing topical or systemic delivery of the main olive polyphenols to the site of interest, which would include both non-pharmaceutical dosage forms as well as nutritional product forms. For example, the extracts can be formulated along with common excipients, diluents, or carriers, and formed into oral tablets, capsules, sprays, mouth washes, lozenges, treated substrates (e.g., oral or topical swabs, pads, or disposable, non-digestible substrate treated with the compositions of the present invention); oral liquids (e. g., suspensions, solutions, emulsions), powders, or any other suitable dosage form.

Non-limiting examples of suitable excipients, diluents, and carriers can be: fillers and extenders such as starch, sugars, mannitol, and silicic derivatives; binding agents such as carboxymethyl cellulose and other cellulose derivatives, alginates, gelatin, and polyvinyl pyrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as acetyl alcohol, glycerol monostearate; adsorptive carriers such as kaolin and bentonite; carriers such as propylene glycol and ethyl alcohol, and lubricants such as talc, calcium and magnesium stearate, and solid polyethyl glycols.

In an embodiment, the composition according to the invention is used for human and/or animal consumption.

In a particular embodiment, the composition is an oral composition.

The compositions are preferably administered as oral dosage forms. For example, the composition may be selected from the group consisting of food compositions, dietary supplements, nutritional compositions, nutraceuticals, powdered nutritional products to be reconstituted in water or milk before consumption, food additives, medicaments, beverages and drinks. Preferred dosage or product forms in this respect include oral tablets, capsules, oral liquids as well as bakery matrices such as bread, cookies and biscuits; and diary products such as butter, cheese, milk and yoghurt.

In yet a particular embodiment, the composition is in the form of a pill, powder, capsule, tablet, gel, beverage, chewing gum, chewable tablet, lozenge or troche.

In another embodiment, the composition is in the form of a powder and then resuspended in a liquid such as water for use.

The extracts described herein can also be formulated as elixirs or solutions for convenient oral administration or as solutions appropriate for parenteral administration, for instance by intramuscular, subcutaneous or intravenous routes. Additionally, the main olive polyphenols are also well suited for formulation as a sustained or prolonged release dosage forms, including dosage forms that release active ingredient only or preferably in a particular part of the intestinal tract, preferably over an extended or prolonged period of time to further enhance effectiveness. The coatings, envelopes, and protective matrices in such dosage forms may be made, for example, from polymeric substances or waxes well known in the (non-)pharmaceutical arts.

### EXAMPLES

### Example 1—Detailed Protocol on Body Parameters Methods

### Study Population & Design

In this study, 62 post-menopausal women (45-70y) were included with a body mass index (BMI)<35 kg/m² in the vicinity of Maastricht (Metabolic Research Unit Maastricht, located at Maastricht University). This study was a randomized, double-blind, controlled parallel trial based on a 12-week supplementation with olive leaves extract compared to control. Participants were randomly assigned to one of two groups: intervention or control. In a parallel design, each individual will follow either a 12-week supplementation (±4 days) with OLE extract (Intervention group) or a 12-week (±4 days) maltodextrin supplementation (control group). Participants underwent a total of four visits: one screening visit and a total of three test days. During screening the eligibility of the potential participant were assessed based on the set in- and exclusion criteria. During Test day 1 (T1) and Test day 3 (T3) the following measurements took place: DXA scan, anthropometric measurements, handgrip test, skin-aging parameters/biophysical properties of the skin, multiple questionnaires and blood draw. During Test day 2 (T2) anthropometric measurements, handgrip test, skin-aging parameters/biophysical properties of the skin, and multiple questionnaires were performed. An overview of the study days and measurements performed during visits is shown in Table 1.

TABLE 1
Overview of measurements taken during visits.

| | Screening | T1 | T2 | T3 |
|---|---|---|---|---|
| Screening Questionnaire | ✓ | | | |
| Anthropometric measurements | ✓ | ✓ | ✓ | ✓ |
| DEXA scan | | ✓ | | ✓ |
| Handgrip test | | ✓ | ✓ | ✓ |
| Skin-aging parameters | | ✓ | ✓ | ✓ |
| Menopause Questionnaire | | ✓ | ✓ | ✓ |
| IPAQ Questionnaire | | ✓ | ✓ | ✓ |
| Blood sample | | ✓ | ✓ | ✓ |

All test days were performed in the morning, in a quiet, temperature-controlled research room. Subjects were instructed to refrain from eating and drinking (except water) from 10 μm in the evening before the test days T1, T2 and T3. Supplements were taken from after T1 till the morning of T3. In addition, subjects were instructed to refrain from any supplement (e.g. multivitamin) in the 4 weeks before the start of the trial and abstain from alcohol 24 hours before and heavy exercise from 48 hours before each test day.

### Administrating of Composition to Subjects

Subjects received a supplement extracted from olive tree leaves named Bonolive®. Bonolive® consist of a mixture of polyphenols derived from olive leaf, standardized for its oleuropein content (40%) (BioActor BV, Maastricht, The Netherlands). The extract was supplied in capsules (Prophar Laboratoire, Anger, France) of identical appearance and flavour compared to the control. The capsules containing both the investigational product and the control were made of cellulose, a compound frequently used in capsule formulations, to improve the contained drugs' dissolution in the gastrointestinal fluids. Each capsule contained 250 mg of the investigational product (olive leaf extract) while the control capsule was maltodextrin.

Subjects were instructed to consume one capsule a day with a glass of water, each morning at breakfast. The 12-week supplementation with OLE started at the end of measurements on T1 and ended the morning of T3. The total daily intake of the investigational product was 250 mg. Other studies with a similar design, duration and study population have also used similar dosages.

### Study Parameters/Endpoints

### Body Composition

Whole-body and regional body composition were estimated using Dual-energy X-ray absorptiometry (DXA) (Horizon® DXA System, Hologic) (52). DXA is considered the current reference technique for assessing muscle mass and body composition in research and clinical practice. Body composition parameters, including regional (left arm, right arm, trunk, left leg, right leg) and total body fat free mass (FFM), fat mass (FM) (g), bone mineral density (BMC) and visceral abdominal tissue (VAT), were obtained. The volume of VAT were calculated using a constant correction factor (0.94 g/cm³). Appendicular lean mass (ALM, in kg) was determined by summing lean mass measures for the arms and legs. ALM index (ALMI=appendicular lean mass (kg)/height (m²)) was also calculated. ALM alone, or scaled to height squared, is the most common metric used as an approximation of muscle mass in sarcopenia research. Fat distribution was assessed by android to gynoid fat mass (kg) ratio (Android/waist to Gynoid/hip Ratio).

### Anthropometric Measures

To further assess body composition, multiple anthropometric measures were performed. Weight, height, calf circumference, waist circumference, arm circumference, hip circumference was assessed. Body mass index (BMI) and hip-waist ratio was afterwards calculated.

### Handgrip Strength

Isometric hand-grip strength was used as a reliable and valid surrogate measure of overall muscular strength. Grip-strength was measured on both hands with a calibrated hand dynamometer (Model SH5001, Saehan). All measures were performed with the participant seated in an upright position, with the arm of the measured hand parallel to the body. Before each measure, the width of the dynamometer's handle was adjusted to each participant's hand size so that the middle phalanges rest on the inner handle. Participants were then instructed to exert maximal force. Each participant performed three consecutive test measurements, alternating between each hand, with ~60-s rest intervals between each measure. All measures were recorded to the nearest 0.5 kg.

### Skin-Aging Parameters/Biophysical Properties of the Skin

A Probes Systems (Courage+Khazaka Electronic, Köln, Germany) was used to assess multiple biophysical properties of the skin. The C+K devices are worldwide used as standard instruments in efficacy testing and claim support of cosmetics as well as food and food supplements. The machine displayed the measurements and numerical result values will be collected and analyzed through a specific software (MPA CTplus).

### Measurement of Skin Moisturization

Skin moisturization was measured using a Corneometer® CM 825 (Courage+Khazaka Electronic, Köln, Germany). The Corneometer® probe measured the capacitance of the stratum corneum using an electric scatter field penetrating the first layers of the stratum corneum (10-20 μm). The capacitance variation of the probe capacitor due to skin surface hydration was measured and skin moisturization was reported in arbitrary units (corneometric units). The measurement was taken in five different points of the right cheek. The selected measurement points delineate the vertices and the center of a quadrangle virtually drawn across the cheek. An increase in the Corneometer value is indicative of a skin moisturizing effect.

### Measurement of Transepidermal Water Loss

Transepidermal water loss (TEWL, perspiratio insensibilis) was measured using a Tewameter® TM 300 (Courage+Khazaka Electronic, Köln, Germany). Tewameter® probe measures indirectly the density gradient of water evaporation over the skin surface using two pairs of sensors (temperature and relative humidity) in an ‘open chamber’ configuration mode. The Fick diffusion law is the basis for the measurement allowing us to calculate the evaporation rate in g·h-1·m-2. The measurement was taken in the center of the right cheek. According to the technical Guide, the scale is as follows: 0-10 (very healthy conditions), 10-15 (healthy conditions), 15-25 (normal condition), 25-30 (affected skin) and >30 (critical condition).

### Measurement of Skin Sebum Content

Skin sebum content was measured using a Sebumeter® SM 815 (Courage+Khazaka Electronic, Köln, Germany) in the center of the forehead. Sebumeter® measurement is based on ‘greasy spot photometry’. When the mat tape of the Sebumeter® cartridge is brought into contact with skin (over a 64 mm² surface) it becomes transparent according to the skin sebum content. The transparency (light transmission) of the mat tape is measured by a photocell which allows to calculate the skin sebum content in μg/cm².

### Measurement of Skin Elasticity and Firmness

Skin elasticity was measured using a Cutometer® MPA 580 (Courage+Khazaka Electronic, Köln, Germany) skin viscoelasticity analyzer. The skin surface of the face (right cheek) was drawn into the aperture (3 mm) of the probe by a negative pressure (450 mbar) for 3 sec and thereafter released for 3 sec. The penetration depth of the skin inside the probe, during the suction and the release phase was measured by a non-contact optical measuring system. R0 and R2 elasticity parameters were measured. R0 is known as the first maximum amplitude of the first suction curve (Uf, skin distensibility), representing the passive behavior of the skin to force. R0 is linked with the stretching of both collagen and elastic fibers and is inversely proportional to their thickness and rigidity. R2 is known as the skin ‘gross’ or ‘overall’ elasticity and is represented by the ratio between the ‘residual deformation’ (Ua) and its maximum ‘distensibility’ (Uf). R2 is a relative elasticity parameter widely used to assess skin elasticity and aging.

### Post-Menopause Symptoms

The Menopause-Specific QOL Questionnaire (MENQOL) (Mapi Research Trust, Lyon, France) is a 29-items (32-items for the intervention questionnaire for use in women taking hormone therapy or Selective estrogen receptor modulators) questionnaire designed to measure four domains of menopausal symptoms (Vasomotor, Psychosocial Physical, Sexual) in the past month or week (two version available).

Hot Flash Interference scale (HFI) (Mapi Research Trust, Lyon, France) is a three-item questionnaire evaluating hot flashes interference with sleep, mood, and concentration in the past two weeks.

### Physical Activity

Physical activity in the previous week was assessed using the self-administered International Physical Activity Questionnaire (IPAQ). The IPAQ assesses duration, frequency and intensity of activities across a range of domains including recreation/leisure, work, transportation and household/yard. Data obtained from the IPAQ-L was used to estimate the total amount of physical activity completed in a 7-day period by weighting the reported minutes per week in each domain by a metabolic equivalent (MET) energy expenditure estimate. MET minutes per week were then calculated by multiplying the duration (minutes), frequency (days) and MET intensity, and summing across the different domains, which include vigorous, moderate and walking, to produce an estimate of total physical activity per week (MET-min/wk.⁻¹). Data on total sitting time was also collected from the IPAQ-L with participants asked to report time spent sitting while at work, at home and leisure-time during the previous 7 days.

### Satiating Capacity

Satiating Capacity was assessed using Haber's scale (a visual analog scale form), ranging from −10 (extreme hunger-painfully hungry) to +10 (extreme satiety—full to nausea). The subjects indicated their level of agreement on hunger or satiety by selecting an appropriate place along the graduated visual scale. All the subjects performed the test every day 30 min before dinner. To facilitate the identification of changes in the feeling of satiety rated using Haber's scale, the full observation period of 90 days was divided into nine 10-day intervals. Within- and between-group differences in satiation (Haber's score) between baseline and these nine times were reported.

### Dietary Habits

To check whether subjects maintained their dietary intake the same during the course of the study, they were asked to complete a 3-day food record on two week days and one weekend day. Dietary intake was measured on two occasions during the study; 1) at the start of the intervention 2) at the end of the intervention period. During test days, the food records were checked, discussed with the subject and completed in case of missing data. Energy and nutrient intake were analyzed using the online dietary assessment tool of The Netherlands Nutrition Centre (www.voedingscentrum.nl), which is based on the Dutch Food Composition Dataset (NEVO, National Institute for Public Health and Environment, Ministry of Health, Welfare and Sport, The Hague, The Netherlands).

### Blood Parameters

Serum lipid profile (total serum cholesterol, triacylglycerol, HDL-cholesterol, LDL-cholesterol), insulin resistance as assessed by Homeostasis Model Assessment (HOMA) using fasting measurements of blood glucose and insulin concentrations, muscle loss as assessed by myostatin and biomarkers of collagen and elastin dynamics.

### Example 2—Effects in Overweight/Obese Subjects

### Methods

In a randomized, double-blind, placebo-controlled study, 77 overweight/obese subjects with total cholesterol levels of 5.0-8.0 mmol/L were randomly assigned to receive 500 mg of olive leaf extract (OLE) or placebo. The olive leaf extract consisted of an optimized mixture of phenolic compounds derived from olive leaf using a 100% water-based extraction method, standardized for its oleuropein content (>16%). Maltodextrin was uses as placebo. Participants were asked to ingest two capsules each morning with 200 ml water 30 minutes before breakfast. The total daily dose of oleuropein within the capsules was 83.5 mg.

### Results

Baseline characteristics of the study population:

TABLE 1
Baseline characteristics

| | Total population | Placebo | OLE |
|---|---|---|---|
| Characteristic | (n = 77) | (n = 38) | (n = 39) |
| Age (years) | 56 ± 10 | 57 ± 9 | 56 ± 11 |
| Gender (Male/Female) | 35/42 | 19/19 | 16/23 |
| BMI (kg/m²) | 29.0 ± 2.7 | 29.6 ± 2.8 | 28.5 ± 2.6 |

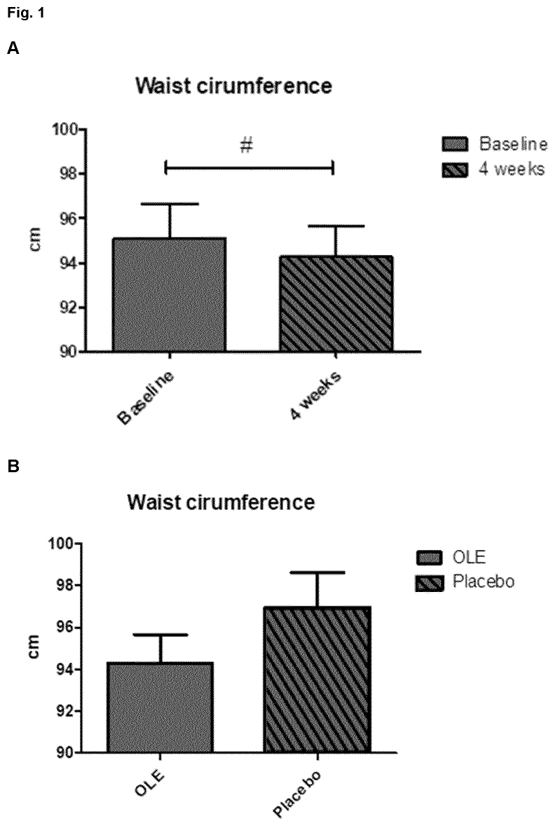

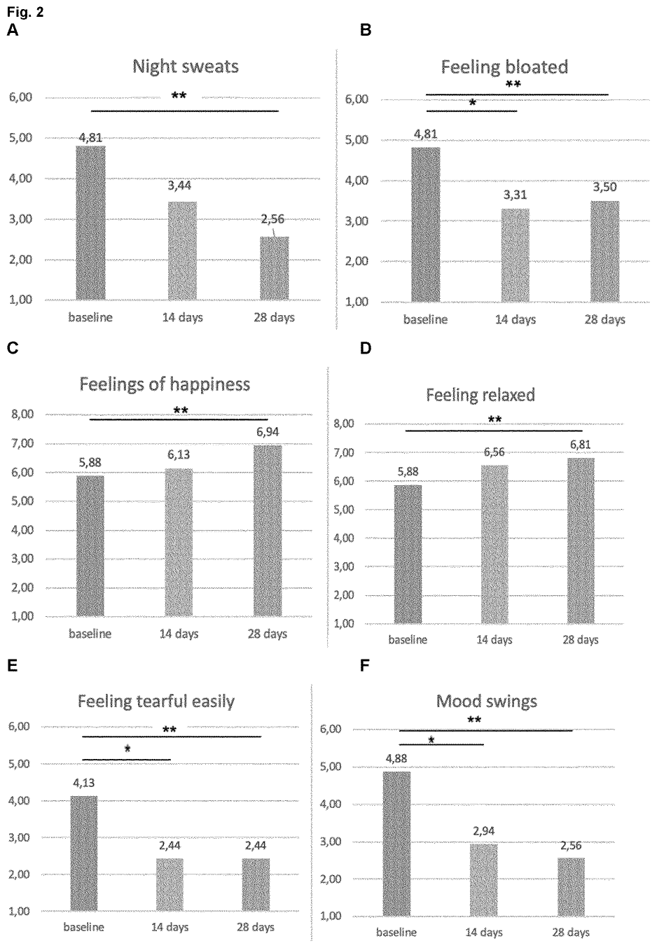

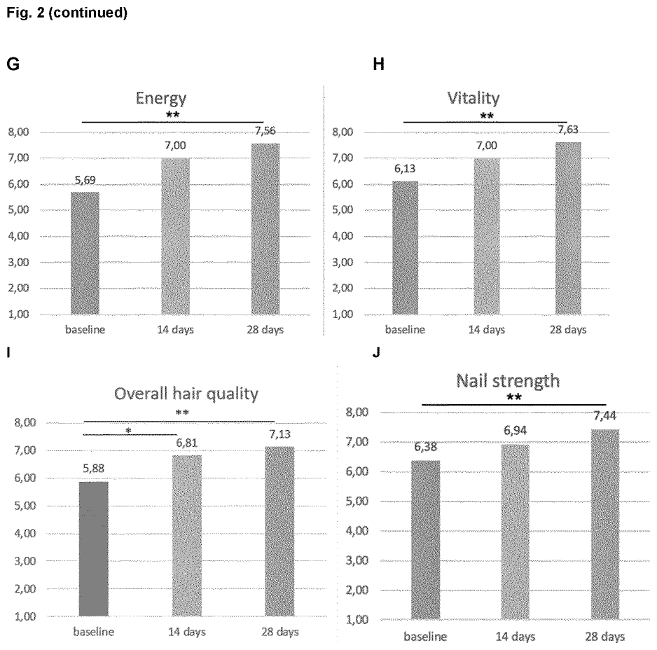

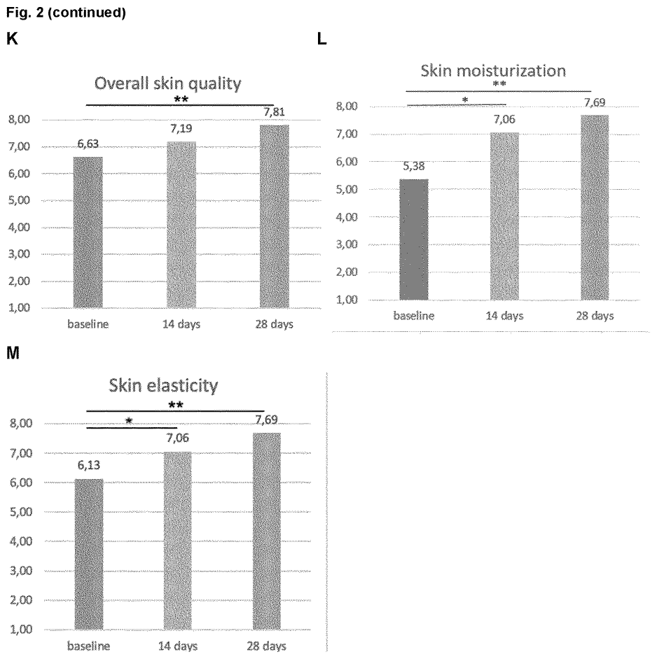

In the group that received olive leaf extracts, there were slightly more women compared to males (16/23). In this group, the waist circumference reduced about 1 cm after 4 weeks of ingestion of olive leaf extract capsules compared to baseline waist circumference (FIG. 1; p trend 0.099). When comparting the exposed group with the control group, a lower waist circumference was observed after 4 weeks of treatment with olive leaf extract (94 cm vs. 97 cm; p=0.22) (FIG. 2).

### Example 3—Effects in Postmenopausal Women

### Methods

Participants were eligible to participate in the study if they were between 45 and 65 years old and were healthy post-menopausal women who did not receive hormone replacement therapy. Subjects were excluded from the study if they were allergic to the olives leaf and/or if they were undergoing any hormone replacement therapy treatments.

The test product was an olive leaf extract (Bonolive®), with a standardize oleuropein content of at least 40%. Each day, for a total duration of 28 days, participants ingested two capsules providing at total of 250 mg of Bonolive® at breakfast.

The study was conducted for a 28-day period, during which three test moments were held (baseline (test 1), day 14 (test 2) and day 28 (test 3). During each one of the test days participants needed to fill out an online questionnaire to assess self-reported post-menopausal symptoms and self-perceived skin and nail quality. Quality of following items perceived in the past two weeks were scored on a 10-point Likert scale ranging from a minimum of 1 “very low/bad” till a maximum of 10 “very high/good”: sleep quality, energy, feelings of happiness, feeling confident, feeling relaxed, vitality, overall skin quality, skin elasticity, skin hydration, overall hair quality, overall hair volume, nail strength. Furthermore, on a scale of 1-10, it was rated how much the subject bothered herself by the following symptoms in the last two weeks. (1=it did not bother me at all; 10=it bothered me a lot): hot flashes, nights sweats, feeling bloated, feeling anxious or nervous, feeling tearful easily, mood swings

### Results

The mean (SD) age of the 16 participating women was 57 (4.3) years. A significant (P≤0.05) improvement was recorded in self-assessed feeling bloated, feeling tearful easily and mood swings following 14 days Bonolive® supplementation when compared to baseline scores. Energy, feelings of happiness, vitality and nail strength scores improved with a high significance (P<0.05) when comparing baseline to 28 days of supplementation.

Following 28 days Bonolive® supplementation, a significant improvement P<0.05) was recorded in self-assessed night sweats, bloating, happiness, relaxation, tearfulness, mood swings, energy, vitality when compared to baseline scores ((FIG. 2A-H). More specifically, a reduction of +2 points on the Likert scale was observed for overall perception of night sweats (4.81 vs 2.56), mood swings (4.88 vs. 2.56) and tearfulness (4.13 vs. 2.44) while overall perception of energy (5.69 vs. 7.56) and vitality (6.13 vs. 7.63) increased with±2 points on the Likert scale. A significant improvement (P≤0.05) after 14 days of supplementation was already recorded for bloating, tearfulness and mood swings.

Self-assessed hair quality (FIG. 2 I) and nail strength (FIG. 2 J) improved significantly after 28 days of supplementation compared to baseline (resp. 5.88 vs. 7.13 for hair quality and 6.38 vs. 7.44 for nail strength).

Overall skin quality was perceived to improve significantly after 28 days of supplementation (FIG. 2 K). For the separate parameters skin moisturization (FIG. 2 L) and skin elasticity (FIG. 2 M), effects were already perceived following 14 days of ingestion of olive leaf extract (P≤0.05). After 28 days of supplementation, an even better improvement was perceived compared to baseline with more than 2 point higher for skin moisturization.

### REFERENCES

Beral V. Breast cancer and hormone-replacement therapy in the Million Women Study. Lancet. 2003; 362 (9382): 419-27.
Ly TTG, Yun J, Lee D H, Chung J S, Kwon S M. Protective Effects and Benefits of Olive Oil and Its Extracts on Women's Health. Nutrients. 2021; 13 (12).
Maartens L W, Leusink G L, Knottnerus J A, Pop V J. Hormonal substitution during menopause: what are we treating? Maturitas. 2000; 34 (2): 113-8.

1-15. (canceled)

16. A method of improving one or more symptoms of menopause or post-menopause in a subject in need thereof, the method comprising administering to the subject an effective dosage of a composition comprising oleuropein and/or an oleuropein metabolite and one or more excipients.

17. The method of claim 16, wherein the improved one or more symptoms are selected from night sweats, mood swings, tearfulness, bloating, fatigue, physical inactivity, hormonal imbalance, sleep disturbance, weight gain, thinning hair, brittle nails, hot flashes, chills, irregular periods, headaches, anxiety, breast soreness, decreased libido, vaginal dryness, vaginal burning, genital atrophy, vulvovaginal atrophy, dysuria, satiating capacity, dietary habits, muscle atrophy, and combinations thereof.

18. The method of claim 16, wherein the improved one or more symptoms of menopause or post-menopause comprise improvement of one or more body composition parameters.

19. The method of claim 18, wherein the one or more body composition parameters are selected from skin-aging parameters, muscle strength parameters, and combinations thereof.

20. The method of claim 19, wherein the one or more body composition parameters are selected from waist circumference, calf circumference, arm circumference, hip circumference, leg circumference, trunk circumference, abdominal circumference, appendicular lean mass, weight, height, total body fat free mass, fat mass, bone mineral density, visceral abdominal tissue, and combinations thereof.

21. The method of claim 18, wherein the skin-aging parameters are selected from skin elasticity, skin moisturization, skin firmness, trans epidermal water loss, skin hydration, skin radiance, skin quality, skin sebum content, skin cellular oxidative stress, wrinkle depth, and combinations thereof.

22. The method of claim 18, wherein the muscle strength parameters are selected from handgrip strength, muscle thermogenesis, muscle contraction, and combinations thereof.

23. The method of claim 16, wherein the composition comprises a standardized amount of oleuropein of from about 5% to about 70% (w/v).

24. The method of claim 16, wherein the composition comprises at least 20 mg of oleuropein.

25. The method of claim 16, wherein the composition is formulated in a daily dosage form comprising from 1 mg/kg to 15 mg/kg of oleuropein.

26. The method of claim 16, wherein the composition comprises oleuropein in combination with one or more additional compounds selected from verbascoside, caffeine, taurine, theanine, inositol, guarana, magnesium, melatonin, ribose, carnitine, co-enzyme Q10, alpha-glycerophosphocholine, alpha-lipoic acid, luteolin-7-glucoside, luteolin, quercetin, and xylose.

27. The method of claim 26, wherein the composition comprises oleuropein and verbascoside.

28. The method of claim 27, wherein the composition comprises from about 0.1% to about 5.0% (w/v) verbascoside.

29. The method of claim 16, wherein the oleuropein metabolite is selected from oleuropein aglycone, hydroxytyrosol, homovanillyl alcohol, isohomovanillyl alcohol, glucuronidated forms thereof, sulfated forms thereof, derivatives thereof, and combinations thereof.

30. The method of claim 16, wherein the composition further comprises a solvent selected from water, ethanol, and combinations thereof.

31. The method of claim 16, wherein the composition is in a form of a pill, a powder, a capsule, a tablet, a gel, a beverage, a chewing gum, a chewable tablet, a lozenge, or a troche.

32. The method of claim 30, wherein the composition comprises at least about 25% alcohol by weight.

33. The method of claim 16, wherein the composition comprises oleuropein and an oleuropein metabolite in a weight ratio of from about 1:1 to about 4:1.

34. The method of claim 16, wherein the composition comprises oleuropein and hydroxytyrosol.

35. The method of claim 34, wherein the weight ratio of oleuropein to hydroxytyrosol is from about 1:1 to about 200:1.
